(19) United States
(12) Patent Application Publication
(10) Pub. No.: US 2017/0112355 A1
(43) Pub. Date: Apr. 27, 2017

(54) IMAGE PROCESSING APPARATUS, IMAGE PROCESSING METHOD, AND COMPUTER-READABLE RECORDING MEDIUM

(71) Applicant: OLYMPUS CORPORATION, Tokyo (JP)

(72) Inventors: Masashi HIROTA, Tokyo (JP); Yamato KANDA, Tokyo (JP); Takashi KONO, Tokyo (JP)

(73) Assignee: OLYMPUS CORPORATION, Tokyo (JP)

(21) Appl. No.: 15/397,321

(22) Filed: Jan. 3, 2017

Related U.S. Application Data
(63) Continuation of application No. PCT/JP2015/067080, filed on Jun. 12, 2015.

(30) Foreign Application Priority Data
Jul. 9, 2014 (JP) 2014-141813

(51) Int. Cl. A61B 1/00; A61B 5/00; A61B 1/045; A61B 1/04

(52) U.S. Cl. CPC A61B 1/00009; A61B 1/043; A61B 5/489; A61B 5/7282; A61B 1/0002; A61B 1/00039; A61B 1/00045; A61B 1/045; A61B 5/0071; A61B 5/0084

(57) ABSTRACT

An image processing apparatus includes: a blood vessel sharpness calculation unit configured to calculate blood vessel sharpness representing sharpness of a visible vascular pattern in a mucosa region in which a mucosa in a lumen is shown in an intraluminal image; an abnormal candidate region extraction unit configured to extract a sharpness reduction region in which the blood vessel sharpness is reduced, as a candidate region for an abnormal region in which the visible vascular pattern is locally lost; and an abnormal region determination unit configured to determine whether the candidate region is the abnormal region based on a shape of the candidate region.

### CROSS REFERENCES TO RELATED APPLICATIONS

This application is a continuation of PCT international application Ser. No. PCT/JP2015/067080, filed on Jun. 12, 2015 which designates the United States, incorporated herein by reference, and which claims the benefit of priority from Japanese Patent Application No. 2014-141813, filed on Jul. 9, 2014, incorporated herein by reference.

### BACKGROUND

1. Technical Field

The disclosure relates to an image processing apparatus, an image processing method, and a computer-readable recording medium, for performing image processing on an intraluminal image of a lumen of a living body.

2. Related Art

There is a known technique of determining whether an abnormal region showing a tumor, or the like, exists in an intraluminal image obtained by imaging the inside of a lumen of a living body, using a medical observation device such as an endoscope and a capsule endoscope. For example, JP 2918162 B1 discloses a technique of calculating shape feature data of a region obtained by binarizing a specific spatial frequency component of an intraluminal image and of determining the presence or absence of an abnormal region by discriminating a blood vessel extending state on the basis of the shape feature data. Hereinafter, the blood vessel extending state will be also referred to as a blood vessel running state. JP 2002-165757 A discloses a technique of setting a region of interest (ROI) on a G-component image among an intraluminal image, calculating feature data by applying a Gabor filter to the ROI, and discriminating abnormality by applying the linear discriminant function to the feature data.

### SUMMARY

In some embodiments, an image processing apparatus includes: a blood vessel sharpness calculation unit configured to calculate blood vessel sharpness representing sharpness of a visible vascular pattern in a mucosa region in which a mucosa in a lumen is shown in an intraluminal image; an abnormal candidate region extraction unit configured to extract a sharpness reduction region in which the blood vessel sharpness is reduced, as a candidate region for an abnormal region in which the visible vascular pattern is locally lost; and an abnormal region determination unit configured to determine whether the candidate region is the abnormal region based on a shape of the candidate region.

In some embodiments, an image processing method is executed by an image processing apparatus for performing image processing on an intraluminal image. The method includes: calculating blood vessel sharpness representing sharpness of a visible vascular pattern in a mucosa region in which a mucosa in a lumen is shown in the intraluminal image; extracting a sharpness reduction region in which the blood vessel sharpness is reduced, as a candidate region for an abnormal region in which the visible vascular pattern is locally lost; and determining whether the candidate region is the abnormal region based on a shape of the candidate region.

In some embodiments, provided is a non-transitory computer-readable recording medium with an executable program stored thereon. The program causes a computer to execute: calculating blood vessel sharpness representing sharpness of a visible vascular pattern in a mucosa region in which a mucosa in a lumen is shown in an intraluminal image; extracting a sharpness reduction region in which the blood vessel sharpness is reduced, as a candidate region for an abnormal region in which the visible vascular pattern is locally lost; and determining whether the candidate region is the abnormal region based on a shape of the candidate region.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram illustrating a configuration of an image processing apparatus according to a first embodiment of the present invention;

FIG. 2 is a flowchart illustrating operation of the image processing apparatus illustrated in FIG. 1;

FIG. 3 is a flowchart illustrating processing of calculating blood vessel sharpness, executed by a blood vessel sharpness calculation unit illustrated in FIG. 1;

FIG. 4 is a schematic diagram illustrating an intraluminal image;

FIG. 5 is a graph illustrating a change in blood vessel sharpness, taken along A-A′ line in FIG. 4;

FIG. 6 is a flowchart illustrating processing of extracting an abnormal candidate region, executed by the abnormal candidate region extraction unit illustrated in FIG. 1;

FIG. 7 is a flowchart illustrating processing of determining an abnormal region, executed by the abnormal region determination unit illustrated in FIG. 1;

FIG. 8 is a schematic diagram for illustrating another example of a structural element setting method;

FIG. 9 is a block diagram illustrating a configuration of a sharpness reduction region extraction unit included in an image processing apparatus according to a modification example 1-1 of the first embodiment of the present invention;

FIG. 10 is a flowchart illustrating processing of extracting an abnormal candidate region, executed by the abnormal candidate region extraction unit including a sharpness reduction region extraction unit illustrated in FIG. 9;

FIG. 11 is a block diagram illustrating a configuration of a sharpness reduction region extraction unit included in an image processing apparatus according to a modification example 1-2 of the first embodiment of the present invention;

FIG. 12 is a flowchart illustrating processing of extracting an abnormal candidate region, executed by the abnormal candidate region extraction unit including a sharpness reduction region extraction unit illustrated in FIG. 11;

FIG. 13 is a block diagram illustrating a configuration of a blood vessel sharpness calculation unit included in an image processing apparatus according to a second embodiment of the present invention;

FIG. 14 is a flowchart illustrating processing of calculating blood vessel sharpness, executed by a blood vessel sharpness calculation unit illustrated in FIG. 13;

FIG. 15 is a block diagram illustrating a configuration of an abnormal candidate region extraction unit included in an image processing apparatus according to a third embodiment of the present invention;

FIG. 16 is a flowchart illustrating processing of extracting an abnormal candidate region, executed by the abnormal candidate region extraction unit illustrated in FIG. 15;

FIG. 17 is a graph illustrating a local change amount of blood vessel sharpness, calculated for an approximate change in blood vessel sharpness, illustrated in FIG. 5; and

FIG. 18 is a diagram illustrating a general configuration of an endoscope system to which the image processing apparatus illustrated in FIG. 1 is applied.

### DETAILED DESCRIPTION

Hereinafter, an image processing apparatus, an image processing method, and an image processing program, according to embodiments of the present invention will be described with reference to the drawings. The present invention is not limited by these embodiments. The same reference signs are used to designate the same elements throughout the drawings.

### First Embodiment

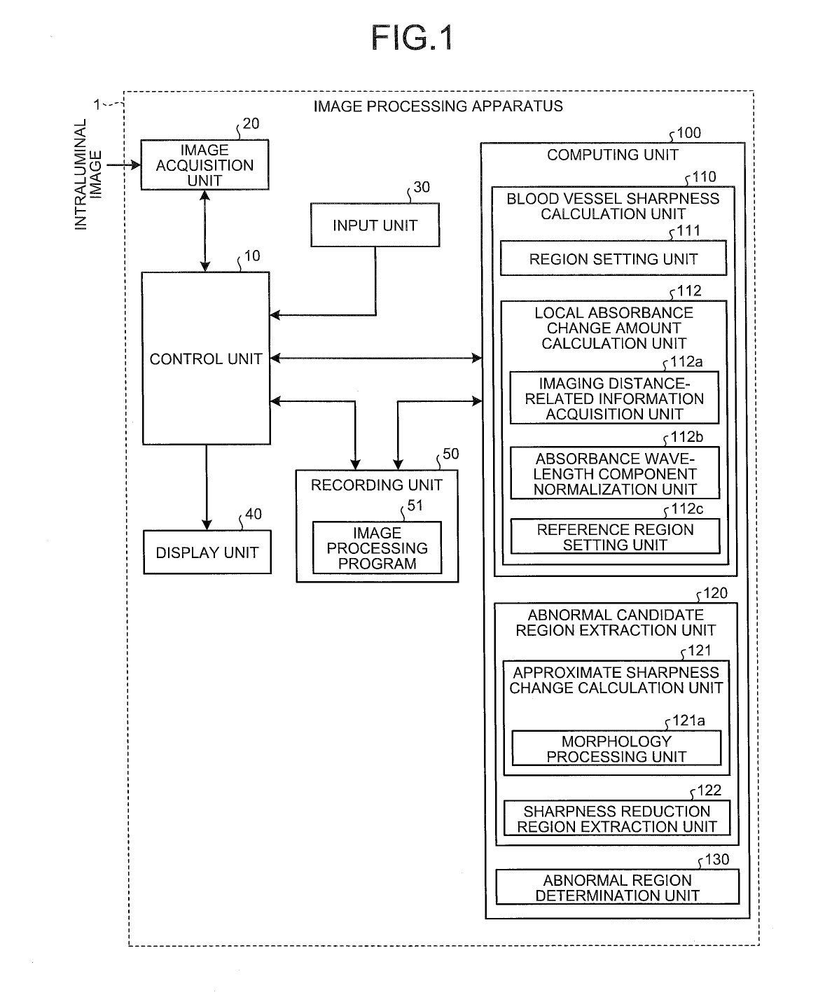

FIG. 1 is a block diagram illustrating a configuration of an image processing apparatus according to a first embodiment of the present invention. An image processing apparatus 1 according to the first embodiment is an apparatus configured to detect an abnormal region as a region of interest including specific characteristics, from an intraluminal image, by performing image processing on an intraluminal image obtained by imaging the inside of a lumen of a living body using a medical observation device such as an endoscope. The typical intraluminal image is a color image having a pixel level (pixel value) for a wavelength component of each of R (red), G (green), and B (blue) at each of pixel positions.

As illustrated in FIG. 1, the image processing apparatus 1 includes a control unit 10, an image acquisition unit 20, an input unit 30, a display unit 40, a recording unit 50, and a computing unit 100. The control unit 10 controls general operation of the image processing apparatus 1. The image acquisition unit 20 obtains image data generated by a medical observation device that has imaged the inside of a lumen. The input unit 30 inputs a signal corresponding to operation from the outside, into the control unit 10. The display unit 40 displays various types of information and images. The recording unit 50 stores image data and various programs obtained by the image acquisition unit 20. The computing unit 100 performs predetermined image processing on the image data.

The control unit 10 is implemented by hardware such as a CPU. The control unit 10 integrally controls general operation of the image processing apparatus 1, specifically, reads various programs recorded in the recording unit 50 and thereby transmitting instruction and data to individual sections of the image processing apparatus 1 in accordance with image data input from the image acquisition unit 20 and with signals, or the like, input from the input unit 30.

The image acquisition unit 20 is configured appropriately in accordance with system modes including a medical observation device. For example, in a case where the medical observation device is connected to the image processing apparatus 1, the image acquisition unit 20 is configured with an interface for incorporating image data generated by the medical observation device. In another case of installing a server for saving image data generated by the medical observation device, the image acquisition unit 20 is configured with a communication device, or the like, connected with the server, and obtains image data by performing data communication with the server. Alternatively, the image data generated by the medical observation device may be transmitted via a portable recording medium. In this case, the portable recording medium is removably attached to the image acquisition unit 20, which is configured with a reader device to read image data of the recorded image.

The input unit 30 is implemented with input devices such as a keyboard, a mouse, a touch panel, and various switches, and outputs input signals generated in response to the external operation of these input devices, to the control unit 10.

The display unit 40 is implemented with display devices such as an LCD and an EL display, and displays various screens including an intraluminal image, under the control of the control unit 10.

The recording unit 50 is implemented with various IC memories such as ROM and RAM as an updatable flash memory, a hard disk that is built in or connected via a data communication terminal, and information recording device such as a CD-ROM and its reading device, and others. The recording unit 50 stores image data of the intraluminal image obtained by the image acquisition unit 20, programs for operating the image processing apparatus 1 and for causing the image processing apparatus 1 to execute various functions, data to be used during execution of this program, or the like. Specifically, the recording unit 50 stores an image processing program 51 that extracts a region in which the visible vascular pattern is locally lost, from an intraluminal image, as an abnormal region, and a threshold table to be used in image processing, or the like.

The computing unit 100 is implemented with a hardware such as a CPU. The computing unit 100 executes image processing of extracting, from an intraluminal image, a region in which the visible vascular pattern is locally lost, as an abnormal region, by reading the image processing program 51.

Next, the configuration of the computing unit 100 will be described. As illustrated in FIG. 1, the computing unit 100 includes a blood vessel sharpness calculation unit 110, an abnormal candidate region extraction unit 120, and an abnormal region determination unit 130. The blood vessel sharpness calculation unit 110 calculates blood vessel sharpness representing sharpness of a visible vascular pattern in a mucosa region in which a mucosa in a lumen is shown in an intraluminal image. The abnormal candidate region extraction unit 120 extracts a sharpness reduction region, that is, a region in which blood vessel sharpness has been reduced, as a candidate region for an abnormal region in which the visible vascular pattern is locally lost. The abnormal region determination unit 130 determines whether the candidate region is an abnormal region on the basis of the shape of the candidate region. Hereinafter, a candidate region for an abnormal region will be referred to as an abnormal candidate region.

A blood vessel existing near the surface of the mucosa is seen through, on the mucosa inside a lumen. An image of such a blood vessel is referred to as a visible vascular pattern. The blood vessel sharpness is a scale of how the visible vascular pattern looks in vividness, clarity, and the level of contrast. In the first embodiment, blood vessel sharpness is set such that the greater the vividness of the visible vascular pattern, the larger the value becomes. In addition, in the present description, “locally lost” represents any of “partially difficult to see” and “partially but completely invisible”.

The blood vessel sharpness calculation unit 110 includes a region setting unit 111 and a local absorbance change amount calculation unit 112. The region setting unit 111 sets a region as a processing target, among an intraluminal image. The local absorbance change amount calculation unit 112 calculates a local absorbance change amount in the region set by the region setting unit 111.

The region setting unit 111 sets a region obtained by eliminating a region in which at least any of mucosa contour, a dark portion, specular reflection, a bubble, and a residue is shown, from the intraluminal image, as a mucosa region to be a calculation target of the local absorbance change amount.

The local absorbance change amount calculation unit 112 calculates the local absorbance change amount of an absorbance wavelength component on the mucosa inside a lumen on the basis of the pixel value of each of the pixels within the mucosa region set by the region setting unit 111, and defines the calculated absorbance change amount as blood vessel sharpness. In the first embodiment, the local absorbance change amount is calculated on the basis of a G-value representing the intensity of the G-component being an absorbance wavelength component inside a lumen, among pixel values of each of the pixels. The local absorbance change amount calculation unit 112 includes an imaging distance-related information acquisition unit 112a, an absorbance wavelength component normalization unit 112b, and a reference region setting unit 112c.

The imaging distance-related information acquisition unit 112a obtains imaging distance-related information, that is, information related to the imaging distance of each of the pixels within the mucosa region. The imaging distance represents a distance from a subject such as a mucosa imaged in an intraluminal image, to an imaging surface of an imaging unit that has imaged the subject.

The absorbance wavelength component normalization unit 112b normalizes a value of an absorbance wavelength component on each of the pixels within the mucosa region on the basis of the imaging distance-related information.

The reference region setting unit 112c sets a pixel range to be referred to in calculating the absorbance change amount, as a reference region, on the basis of the imaging distance-related information. Specifically, the closer view the image becomes, the thicker the blood vessels are likely to appear on the intraluminal image. Accordingly, the reference region is set such that the closer view the image becomes, the greater the reference region.

The abnormal candidate region extraction unit 120 includes an approximate sharpness change calculation unit 121 and a sharpness reduction region extraction unit 122. The approximate sharpness change calculation unit 121 calculates the approximate change in the blood vessel sharpness calculated by the blood vessel sharpness calculation unit 110. The sharpness reduction region extraction unit 122 extracts, from the approximate change in the blood vessel sharpness, a sharpness reduction region, that is, the region in which the blood vessel sharpness is reduced on the visible vascular pattern. Among these, the approximate sharpness change calculation unit 121 includes a morphology processing unit 121a, and calculates the approximate change in the blood vessel sharpness by performing grayscale morphology processing for handling grayscale images, on the blood vessel sharpness. The sharpness reduction region extraction unit 122 performs threshold processing on the approximate change in the blood vessel sharpness, thereby extracting a sharpness reduction region. This sharpness reduction region is output as an abnormal candidate region.

The abnormal region determination unit 130 incorporates the abnormal candidate region extracted by the abnormal candidate region extraction unit 120 and determines whether the abnormal candidate region is an abnormal region on the basis of the circular degree of the abnormal candidate region. Specifically, in a case where the abnormal candidate region is substantially circular, the abnormal candidate region is determined as an abnormal region.

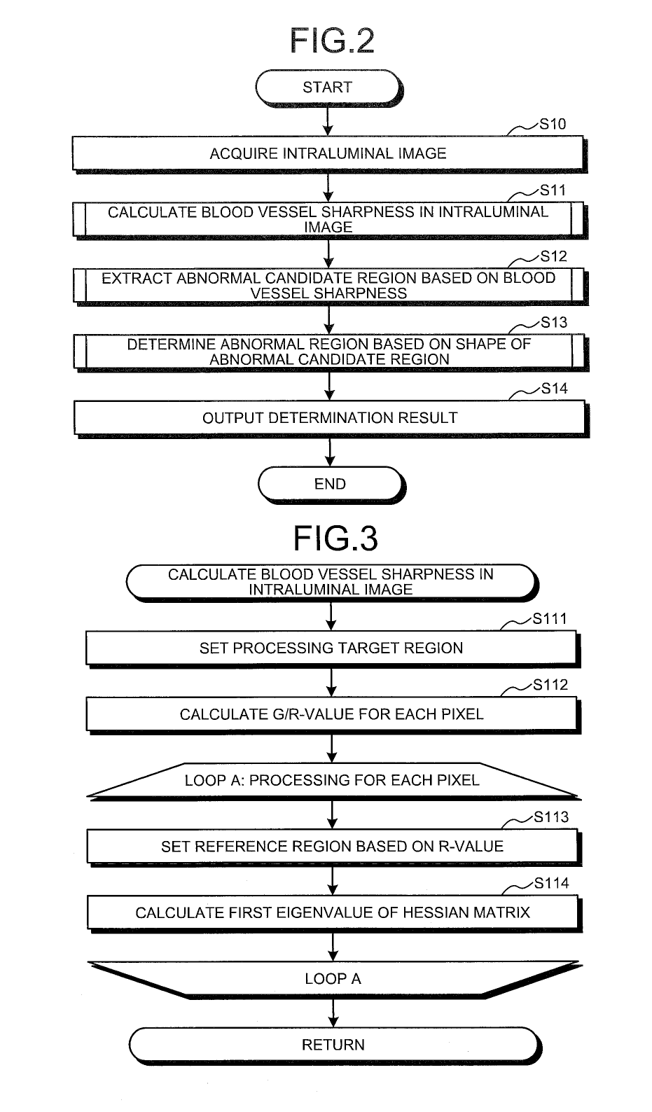

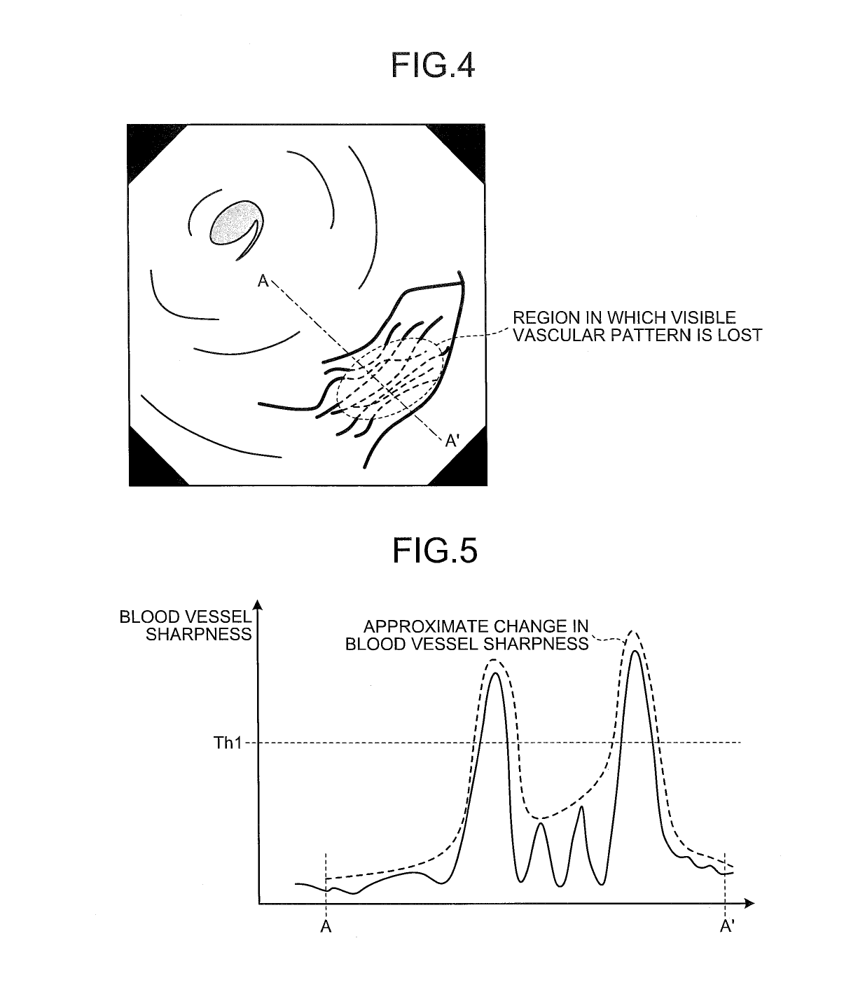

Next, operation of the image processing apparatus 1 will be described. FIG. 2 is a flowchart illustrating operation of the image processing apparatus 1. First, in step S10, the image processing apparatus 1 acquires an intraluminal image via the image acquisition unit 20. In the first embodiment, an intraluminal image is generated by imaging in which illumination light (white light) including wavelength components of R, G, and B is emitted inside a lumen using an endoscope. The intraluminal image has pixel values (R-value, G-value, and B-value) that correspond to these wavelength components on individual pixel positions. FIG. 4 is a schematic diagram illustrating an exemplary intraluminal image obtained in step S10.

In subsequent step S11, the computing unit 100 incorporates the intraluminal image and calculates blood vessel sharpness of the intraluminal image. The blood vessel sharpness can be represented as an absorbance change amount in a blood vessel region. Accordingly, the first embodiment calculates a first eigenvalue (maximum eigenvalue) in a Hessian matrix of the pixel value of each of the pixels within the intraluminal image, as an absorbance change amount.

FIG. 3 is a flowchart illustrating processing of calculating blood vessel sharpness, executed by the blood vessel sharpness calculation unit 110. In step S111, the region setting unit 111 sets a region obtained by eliminating a region in which any of mucosa contour, a dark portion, specular reflection, a bubble, and a residue is shown, from the intraluminal image, that is, sets a mucosa region, as a processing target region. Specifically, the region setting unit 111 calculates a G/R-value for each of the pixels within the intraluminal image, and sets a region whose G/R-value is equal to or less than a threshold, that is, a reddish region, as a processing target region.

The method for setting the processing target region is not limited to the above-described method. Various known methods may be applied. For example, as disclosed in JP 2007-313119 A, it is allowable to detect a bubble region by detecting a match between a bubble model to be set on the basis of characteristics of a bubble image, such as an arc-shaped protruding edge due to illumination reflection, existing at a contour portion of a bubble or inside the bubble, with an edge extracted from the intraluminal image. As disclosed in JP 2011-234931 A, it is allowable to extract a black region on the basis of color feature data based on each of the pixel values (R-value, G-value, and B-value) and to determine whether the black region is a dark portion on the basis of the direction of the pixel value change around this black region. Alternatively, it is allowable to extract a white region on the basis of color feature data based on each of the pixel values and to determine whether the white region is a specular reflection region on the basis of the pixel value change around a boundary of the white region. Further alternatively, it is allowable to detect a residue candidate region, that is assumed to be a non-mucosa region, on the basis of color feature data based on each of the pixel values and to determine whether the residue candidate region is a mucosa region on the basis of the positional relationship between the residue candidate region and the edge extracted from the intraluminal image.

In subsequent step S112, the local absorbance change amount calculation unit 112 calculates a G/R-value for each of the pixels within the processing target region, set in step S111. The R-component of the illumination light corresponds to a wavelength band with very little absorption for hemoglobin. Accordingly, the attenuation amount of the R-component inside a lumen corresponds to the distance for which the illumination light is transmitted through the inside of the lumen. Therefore, in the first embodiment, the R-value for each of the pixels within the intraluminal image is used as imaging distance-related information on the corresponding pixel position. The shorter the imaging distance, that is, the closer view the subject becomes, the greater the R-value. The longer the imaging distance, that is, the more distant the subject becomes, the smaller the R-value. Accordingly, the G/R-value can be determined as a value obtained as a result of normalizing the G-component being the absorbance wavelength component inside the lumen, by the imaging distance.

Subsequently, the local absorbance change amount calculation unit 112 calculates a local absorbance change amount on each of the pixels by executing loop-A processing for each of the pixels within the processing target region.

In step S113, the reference region setting unit 112c sets a reference region that is a range of pixels to be referred to in calculating the local absorbance change amount on the basis of the R-value on the processing target pixel. Note that the closer view the image becomes, the thicker the blood vessels are likely to appear on the intraluminal image. Accordingly, it is necessary to set the reference region adaptively in accordance with the imaging distance. Accordingly, the reference region setting unit 112c sets the reference region such that the closer view the subject becomes on the processing target pixel, the greater the reference region becomes, on the basis of the R-value having a correlation with the imaging distance. In actual processing, a table associating the R-value with the reference region is created and recorded in the recording unit 50 beforehand, and the reference region setting unit 112c sets a reference region according to the R-value, for each of the pixels, with reference to the table.

In subsequent step S114, the local absorbance change amount calculation unit 112 calculates a first eigenvalue (maximum eigenvalue) of the Hessian matrix indicated in the next formula (1) using a G/R-value calculated for the processing target pixel and the surrounding pixel within the reference region.

$$H ( {x}_{0} , {y}_{0} ) = ( \begin{matrix}\frac{{\partial }^{2} I ( {x}_{0} , {y}_{0} )}{\partial {x}^{2}} & \frac{{\partial }^{2} I ( {x}_{0} , {y}_{0} )}{\partial x \partial y} \\ \frac{{\partial }^{2} I ( {x}_{0} , {y}_{0} )}{\partial y \partial x} & \frac{{\partial }^{2} I ( {x}_{0} , {y}_{0} )}{\partial {y}^{2}}\end{matrix} ) \qquad ( 1 )$$

The value I (x₀, y₀) in Formula (1) represents a G/R-value of a pixel positioned on coordinates (x₀, y₀) within the intraluminal image.

The first eigenvalue of the above-described Hessian matrix H (x₀, y₀) represents a maximum principal curvature (curvedness) at a portion surrounding the processing target pixel. Accordingly, the first eigenvalue can be determined as a local absorbance change amount. The local absorbance change amount calculation unit 112 outputs the local absorbance change amount as blood vessel sharpness at the corresponding pixel position. Note that, while the first embodiment calculates the first eigenvalue of the Hessian matrix as the blood vessel sharpness, the present invention is not limited to this. It is also allowable to calculate the blood vessel sharpness using known modulation transfer function (MTF) and a contrast transfer function (CTF).

After the loop-A processing has been performed for all the pixels within the processing target region, operation of the computing unit 100 returns to the main routine.

In step S12 subsequent to step S11, the abnormal candidate region extraction unit 120 extracts an abnormal candidate region on the basis of the blood vessel sharpness that is, the local absorbance change amount, calculated in step S11.

FIG. 5 is a graph illustrating a change in blood vessel sharpness, taken along A-A′ line in FIG. 4. In the first embodiment, the abnormal candidate region is a region in which local loss of the visible vascular pattern is suspected. As illustrated in FIGS. 4 and 5, these regions appear on the intraluminal image, as the region with low blood vessel sharpness. Accordingly, the abnormal candidate region extraction unit 120 extracts an abnormal candidate region by detecting the region in which the blood vessel sharpness is reduced.

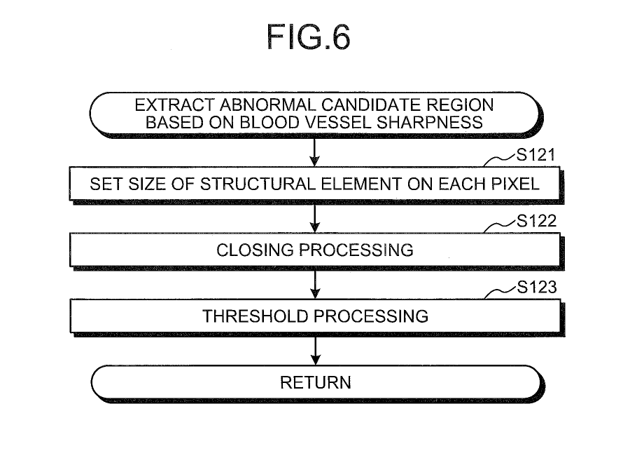

FIG. 6 is a flowchart illustrating processing of extracting an abnormal candidate region, executed by the abnormal candidate region extraction unit 120. In step S121, the approximate sharpness change calculation unit 121 sets the size of a structural element of each of the pixels to be used at calculation of the approximate change in the blood vessel sharpness. Note that the closer view the image becomes, the larger the region in which the visible vascular pattern has lost is likely to be imaged. Accordingly, it is necessary to set the size of the structural element adaptively in accordance with the imaging distance. Accordingly, the approximate sharpness change calculation unit 121 obtains an R-value having correlation with the imaging distance and sets the size of the structural element such that the greater the R-value, that is, the shorter the imaging distance, the greater the size of the structural element.

In subsequent step S122, the morphology processing unit 121a calculates the approximate change in the blood vessel sharpness by performing closing processing of morphology on the blood vessel sharpness calculated in step S11 using the structural element with the size that has been set in accordance with the R-value of each of the pixels (refer to FIG. 5).

In subsequent step S123, the sharpness reduction region extraction unit 122 performs threshold processing on the approximate change in the blood vessel sharpness calculated in step S122, and extracts a region in which the blood vessel sharpness is equal to or less than a predetermined threshold Th1, as an abnormal candidate region. Thereafter, operation of the computing unit 100 returns to the main routine.

In step S13 subsequent to step S12, the abnormal region determination unit 130 performs determination of the abnormal region on the basis of the shape of the abnormal candidate region extracted in step S12. Note that the abnormal candidate region includes not only the region having blood vessel sharpness that has been reduced due to loss of the visible vascular pattern, but also a normal mucosa region in which blood vessels are not clearly seen. These mucosa regions have characteristics in shapes including having a large area, unlike the abnormal region in which visible vascular patterns have been locally lost. Accordingly, the abnormal region determination unit 130 determines whether the abnormal candidate region is an abnormal region on the basis of the characteristics in the shapes.

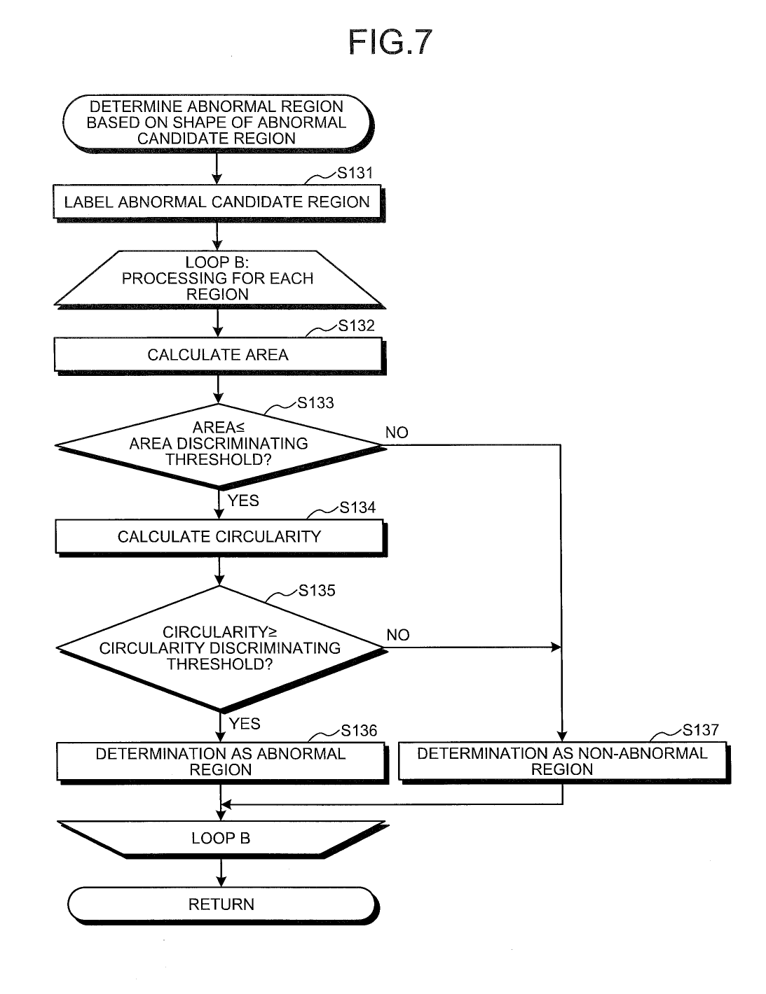

FIG. 7 is a flowchart illustrating processing of determining an abnormal region, executed by the abnormal region determination unit 130. In step S131, the abnormal region determination unit 130 labels the abnormal candidate region extracted from the intraluminal image.

Subsequently, the abnormal region determination unit 130 performs loop-B processing on each of the regions labeled in step S131.

First, in step S132, the area of the processing target region, namely, the area of the abnormal candidate region is calculated. Specifically, the number of pixels included in the region is counted.

In subsequent step S133, the abnormal region determination unit 130 determines whether the area calculated in step S132 is equal to or less than the threshold for discriminating the area (area discriminating threshold). In a case where the calculated area is larger than the area discriminating threshold (step S133: No), the abnormal region determination unit 130 determines that the region is not an abnormal region, that is, determines it is a non-abnormal region (step S137).

In contrast, in a case where the area is equal to or less than the area discriminating threshold (step S133: Yes), the abnormal region determination unit 130 subsequently calculates circularity of the processing target region (step S134). The circularity is a scale representing how circular the shape of the region is, and is provided as 4 πS/L² in a case where the area of the region is S, and the circumference length is L. The closer to 1 the value of circularity is, the closer to a perfect circle the shape of the region is. Note that it is allowable to use a scale other than the circularity as long as it is a scale indicating how circular the shape of the abnormal candidate region is.

In subsequent step S135, the abnormal region determination unit 130 determines whether the circularity calculated in step S134 is equal to or more than a threshold for discriminating the circularity (circularity discriminating threshold). If the calculated circularity is less than the circularity discriminating threshold (step S135: No), the abnormal region determination unit 130 determines that the region is not an abnormal region, i.e., the region is a non-abnormal region (step S137).

In contrast, if the circularity is equal to or more than the circularity discriminating threshold (step S135: Yes), the abnormal region determination unit 130 determines that the processing target region is an abnormal region (step S136).

After the loop-B processing has been performed on all the regions labeled in step S131, operation of the computing unit 100 returns to the main routine.

In step S14 subsequent to step S13, the computing unit 100 outputs a determination result in step S13. In response to this, the control unit 10 displays the region determined as an abnormal region, onto the display unit 40. The method for displaying the region determined as an abnormal region is not particularly limited. An exemplary method would be to superpose a mark indicating the region determined as an abnormal region, onto the intraluminal image and to display the region determined to be an abnormal region in a color different from other regions, or with shading. Together with this, the determination result of the abnormal region in step S13 may be recorded on the recording unit 50. Thereafter, operation of the image processing apparatus 1 is finished.

As described above, according to the first embodiment of the present invention, the region in which the absorbance change amount is locally reduced is extracted as an abnormal candidate region, from the intraluminal image, and whether the abnormal candidate region is an abnormal region is determined on the basis of the shape of the abnormal candidate region. With this configuration, it is possible to extract, with high accuracy, the region in which the visible vascular pattern has been locally lost.

Note that, while the above-described first embodiment calculates the first eigenvalue of the Hessian matrix as the absorbance change amount, the method for calculating the absorbance change amount is not limited to this. For example, it is allowable to apply a band-pass filter to the pixel value of each of the pixels within the intraluminal image. In this case, it would be sufficient to adaptively set the filter size on the basis of the R-value of the processing target pixel. Specifically, it would be preferable to set such that the smaller the R-value, that is, the longer the imaging distance, the larger the filter size.

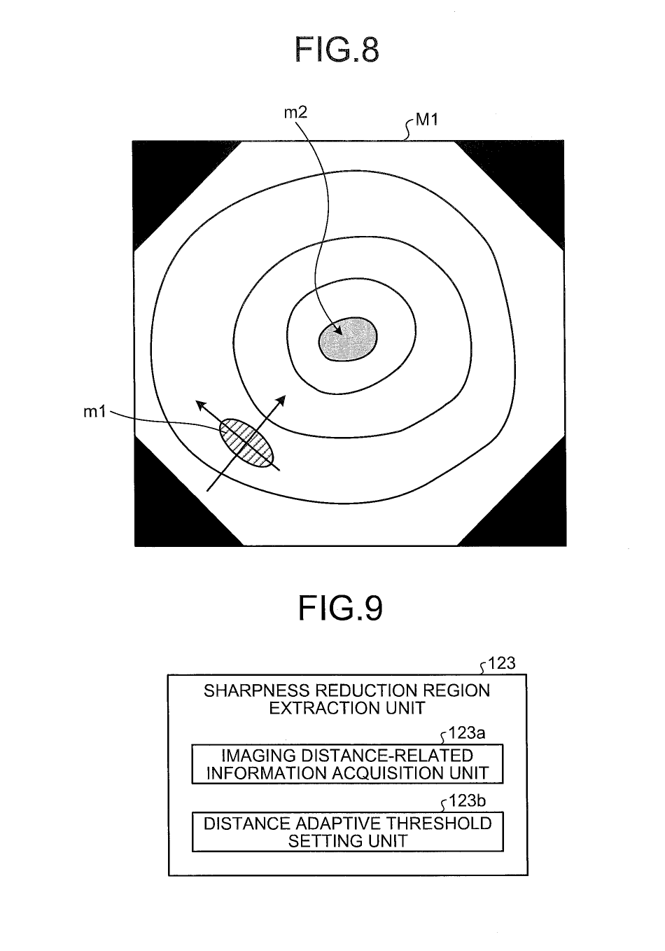

Moreover, the above-described first embodiment sets the size of the structural element used in morphology processing on the basis of the imaging distance. In addition to this, it is allowable to set the shape and orientation of the structural element. FIG. 8 is a schematic diagram for illustrating another example of a structural element setting method.

In the case of imaging the inside of a lumen by an endoscope, the imaging direction corresponds to a slanting direction with respect to the mucosa surface as a subject, in many cases. In this case, the size of the subject in the depth direction viewed from the endoscope appears smaller, on the image, compared with the case in which the same subject is imaged from the front. Accordingly, the shape and the orientation of the structural element is set such that its size becomes small in a direction where the mucosa surface inclination with respect to the imaging surface is maximum, that is, in a direction where an actual change in the imaging distance is greater with respect to the distance on the intraluminal image, and such that its size becomes great in a direction orthogonal to the direction where the change in the imaging distance is greater. With this setting, it is possible to perform morphology processing appropriately. For example, when imaging is performed toward the depth direction of the lumen as illustrated in an image Ml in FIG. 8, the shape and the orientation of a structural element m1 are set such that the direction starting from each of the positions within the image toward a deep portion m2 of the lumen is a short-axis direction of an ellipse, and that the direction orthogonal to the direction toward the deep portion m2 is a long-axis direction of the ellipse.

Also note that, while the above-described first embodiment performs determination of an abnormal region by sequentially comparing the area and circularity of an abnormal candidate region with a threshold, the determination method is not limited to this as long as it is possible to perform determination on the basis of the area and circularity of the abnormal candidate region. For example, it is allowable to perform determination about circularity first. Alternatively, it is allowable to preliminarily create a table on which both the area and the circularity can be referred to, and to simultaneously evaluate the area and circularity calculated for this abnormal candidate region.

### Modification Example 1-1

Next, a modification example 1-1 of the first embodiment of the present invention will be described. FIG. 9 is a block diagram illustrating a configuration of a sharpness reduction region extraction unit included in a computing unit of an image processing apparatus according to the modification example 1-1. On the computing unit 100 (refer to FIG. 1) of an image processing apparatus according to the modification example 1-1, the abnormal candidate region extraction unit 120 includes a sharpness reduction region extraction unit 123 illustrated in FIG. 9 instead of the sharpness reduction region extraction unit 122. Note that individual configurations and operation of the computing unit 100 other than the sharpness reduction region extraction unit 123 and individual configurations and operation of the image processing apparatus 1 are similar to the case of the first embodiment.

The sharpness reduction region extraction unit 123 includes an imaging distance-related information acquisition unit 123a and a distance adaptive threshold setting unit 123b. The imaging distance-related information acquisition unit 123a obtains an R-value of each of the pixels, as information regarding an imaging distance between a subject shown in the intraluminal image and an imaging surface of the imaging unit that has imaged the subject. The distance adaptive threshold setting unit 123b adaptively sets a threshold (refer to FIG. 5) to be used for extracting a sharpness reduction region from the approximate change in the blood vessel sharpness, in accordance with the R-value.

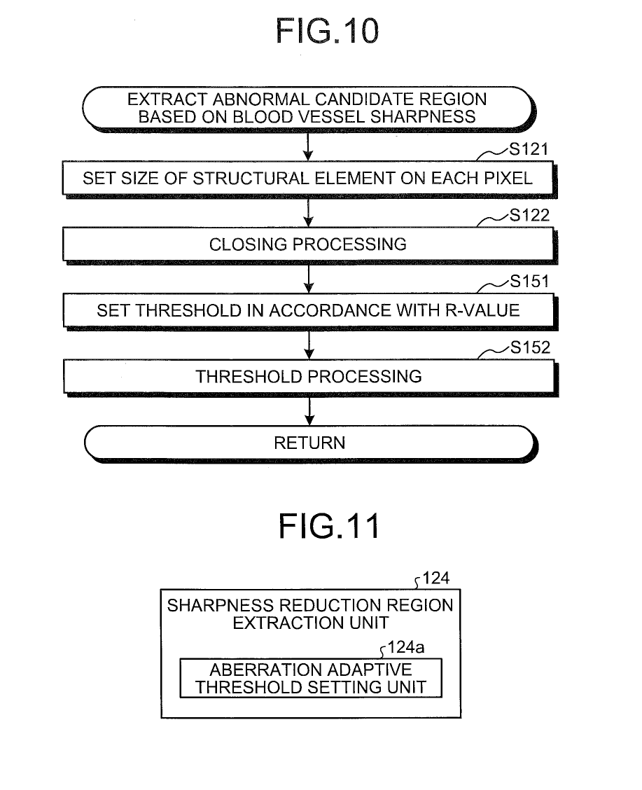

General operation of the image processing apparatus according to the modification example 1-1 is similar to the case of the first embodiment, except for a difference in details of extraction processing of the abnormal candidate region illustrated in FIG. 2 (step S12) from the first embodiment. FIG. 10 is a flowchart illustrating processing of extracting an abnormal candidate region, executed by the abnormal candidate region extraction unit including the sharpness reduction region extraction unit 123. Note that steps S121 and S122 illustrated in FIG. 10 are similar to the steps in the first embodiment.

In step S151 subsequent to step S122, the sharpness reduction region extraction unit 123 adaptively sets a threshold for extracting a region in which the blood vessel sharpness has been reduced, in accordance with the R-value of each of the pixels within the processing target region (refer to step S111 in FIG. 3) that has been set on an intraluminal image.

Note that, in a region deviated from the depth of field of the imaging unit in imaging the inside of a lumen, blood vessel sharpness would be more reduced than the other regions even when it is not an abnormal region. To cope with this, the sharpness reduction region extraction unit 123 obtains an R-value having correlation with the imaging distance, and sets such that the more the R-value deviates from a predetermined range, specifically, from a range corresponding to the depth of field, the smaller the threshold. In actual processing, a table associating the R-value with the threshold is created on the basis of the depth of field and recorded in the recording unit 50 beforehand, and the distance adaptive threshold setting unit 123b sets a threshold for each of the pixels according to the R-value with reference to this table.

In subsequent step S152, the sharpness reduction region extraction unit 123 performs threshold processing on the approximate change in the blood vessel sharpness using a threshold set for each of the pixels in step S151, thereby extracting a region in which the blood vessel sharpness is equal to or less than the threshold, as an abnormal candidate region. Thereafter, operation of the computing unit 100 returns to the main routine.

As described above, according to the modification example 1-1, the threshold used in extracting the sharpness reduction region is adaptively set in accordance with the imaging distance. With this configuration, it is possible to suppress erroneous detection of the sharpness reduction region in the region deviated from the depth of field, among the intraluminal image.

### Modification Example 1-2

Next, a modification example 1-2 of the first embodiment of the present invention will be described. FIG. 11 is a block diagram illustrating a configuration of a sharpness reduction region extraction unit included in a computing unit of an image processing apparatus according to the modification example 1-2. On the computing unit 100 (refer to FIG. 1) of an image processing apparatus according to the modification example 1-2, the abnormal candidate region extraction unit 120 includes a sharpness reduction region extraction unit 124 illustrated in FIG. 11 instead of the sharpness reduction region extraction unit 122. Note that individual configurations and operation of the computing unit 100 other than the sharpness reduction region extraction unit 124 and individual configurations and operation of the image processing apparatus 1 are similar to the case of the first embodiment.

The sharpness reduction region extraction unit 124 includes an aberration adaptive threshold setting unit 124a and extracts a sharpness reduction region by performing threshold processing using a threshold set by the aberration adaptive threshold setting unit 124a. The aberration adaptive threshold setting unit 124a is an optical system adaptive threshold setting unit that adaptively sets a threshold in accordance with characteristics of an optical system included in an endoscope, or the like, that has imaged the inside of a lumen. In the modification example 1-2, the aberration adaptive threshold setting unit 124a sets a threshold in accordance with the coordinates of each of the pixels within the intraluminal image so as to reduce the effects of the aberration of the optical system, as an example of the characteristics of the optical system.

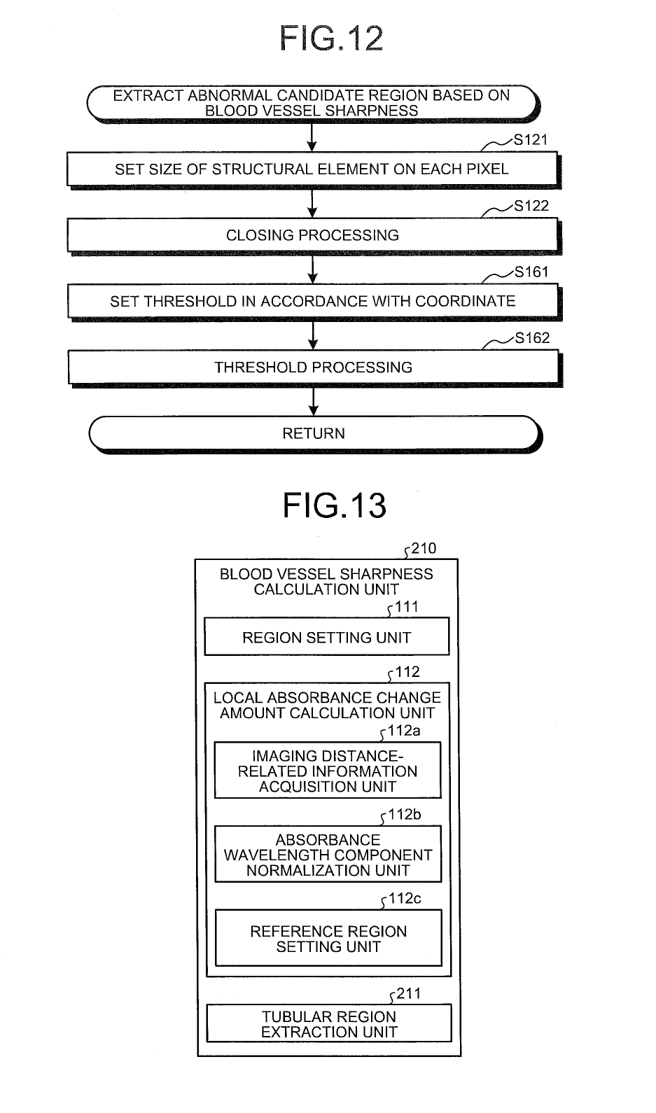

General operation of the image processing apparatus according to the modification example 1-2 is similar to the case of the first embodiment, except for a difference in details of extraction processing of the abnormal candidate region illustrated in FIG. 2 (step S12) from the first embodiment. FIG. 12 is a flowchart illustrating processing of extracting an abnormal candidate region, executed by the abnormal candidate region extraction unit including the sharpness reduction region extraction unit 124. Note that steps S121 and S122 illustrated in FIG. 12 are similar to the steps in the first embodiment.

In step S161 subsequent to step S122, the sharpness reduction region extraction unit 124 adaptively sets a threshold for extracting the region with a reduced blood vessel sharpness in accordance with the coordinates of each of the pixels within the processing target region (refer to step S111 in FIG. 3) that has been set on an intraluminal image.

The intraluminal image includes a region in which blur is likely to occur due to effects of the optical system included in the endoscope, or the like. Specifically, blur is likely to arise in a region having a great level of aberration such as spherical aberration, coma aberration, astigmatism, and field curvature, that is, in a peripheral region of the intraluminal image. In these regions, sharpness reduction regions might be erroneously detected because the blood vessel sharpness is more reduced than the other regions even in a region that is not an abnormal region.

Accordingly, the aberration adaptive threshold setting unit 124a sets the threshold such that the greater the effects of aberration in the region, the smaller the threshold, on the basis of the coordinates of each of the pixels of the intraluminal image. In actual processing, a table associating the coordinates of each of the pixels of the intraluminal image with the threshold is created and recorded in the recording unit 50 beforehand, and the aberration adaptive threshold setting unit 124a sets a threshold according to the coordinates, for each of the pixels, with reference to this table.

In subsequent step S162, the sharpness reduction region extraction unit 124 performs threshold processing on the approximate change in the blood vessel sharpness using a threshold set for each of the pixels in step S161, thereby extracting a region in which the blood vessel sharpness is equal to or less than the threshold, as an abnormal candidate region. Thereafter, operation of the computing unit 100 returns to the main routine.

As described above, according to the modification example 1-2, the threshold to be used in extraction of the sharpness reduction region is adaptively set in accordance with the coordinates of the pixel. Accordingly, it is possible to enhance accuracy in detecting the sharpness reduction region even in a region in which effects of aberration is significant, or the like.

### Modification Example 1-3

Next, Modification Example 1-3 of the first embodiment of the present invention will be described. The threshold used for extracting a sharpness reduction region may be set on the basis of both the imaging distance and the coordinates, corresponding to each of the pixels within the intraluminal image. In actual processing, it is sufficient to create a table associating the imaging distance/pixel coordinates with the threshold beforehand and to record the table in the recording unit 50.

In this case, it is possible to enhance accuracy in detecting the sharpness reduction region even for the region that deviates from the depth of field and that has significant effects of aberration of the optical system.

It is allowable to set the threshold to be used for extracting the sharpness reduction region in accordance with various elements other than these. For example, in a case of using an endoscope capable of switching focal length of the optical system, it is allowable to set the threshold on the basis of the depth of field that changes with the focal length. In actual processing, a plurality of types of tables associating the R-value as imaging distance-related information with the threshold on the basis of the depth of field (refer to modification example 1-1) are prepared in accordance with the switchable focal length. The table selection is performed on the basis of focal length information at the imaging of the intraluminal image as a processing target, and the threshold is set for each of the pixels, using the selected table. Additionally, the focal length information may be directly input from the endoscope, or the like, into the image processing apparatus, or the focal length information at the time of imaging may be associated with the image data of the intraluminal image, and the focal length information may be incorporated together when the image processing apparatus 1 acquires the intraluminal image.

### Second Embodiment

Next, a second embodiment of the present invention will be described. FIG. 13 is a block diagram illustrating a configuration of a blood vessel sharpness calculation unit included in an image processing apparatus according to the second embodiment. On the image processing apparatus according to the second embodiment, the computing unit 100 (refer to FIG. 1) includes a blood vessel sharpness calculation unit 210 illustrated in FIG. 13 instead of the blood vessel sharpness calculation unit 110. Note that individual configurations and operation of the computing unit 100 other than the blood vessel sharpness calculation unit 210 and individual configurations and operation of the image processing apparatus 1 are similar to the case of the first embodiment.

The blood vessel sharpness calculation unit 210 further includes a tubular region extraction unit 211 in addition to the region setting unit 111 and the local absorbance change amount calculation unit 112. The tubular region extraction unit 211 extracts a tubular region having a tubular shape, from the intraluminal image, on the basis of the pixel value of each of the pixels within the intraluminal image.

Next, operation of the image processing apparatus according to a second embodiment will be described. General operation of the image processing apparatus according to the second embodiment is similar to the case of the first embodiment (refer to FIG. 2), except for a difference in details of processing of calculating the blood vessel sharpness in step S11, from the first embodiment.

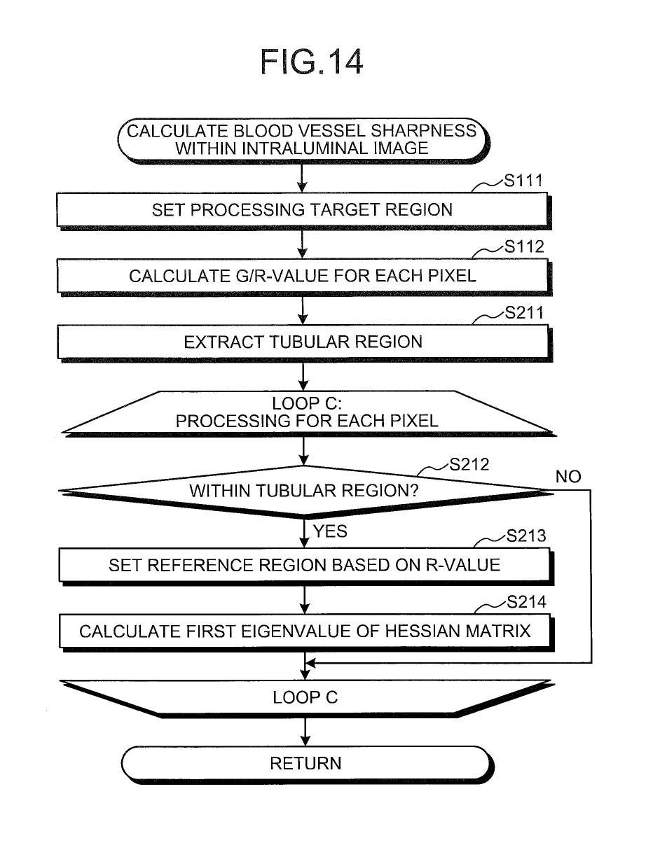

FIG. 14 is a flowchart illustrating processing of calculating blood vessel sharpness, executed by the blood vessel sharpness calculation unit 210. Note that steps S111 and S112 illustrated in FIG. 14 are similar to the steps in the first embodiment (refer to FIG. 3).

In step S211 subsequent to step S112, the tubular region extraction unit 211 extracts a tubular region from the processing target region on the basis of the pixel value of the pixel within the processing target region, set in step S111. In detail, the tubular region extraction unit 211 calculates a shape index on the basis of the pixel value of each of the pixels within the processing target region, and executes threshold processing on the shape index, thereby extracting a tubular region. A shape index SI is given by the following formula (2) using a first eigenvalue eVal_1 and a second eigenvalue eVal_2 (eVal_1>eVal_2), of the Hessian matrix.

$$SI = \frac{2}{π} arc tan ( \frac{eVal \_ 2 + eVal \_ 1}{eVal \_ 2 - eVal \_ 1} ) \qquad ( 2 )$$

For example, it would be preferable to extract a region in which the shape index SI given by Formula (2) is equal to or less than −0.4, that is, a region having a recess shape, as a tubular region.

Subsequently, the blood vessel sharpness calculation unit 210 calculates a local absorbance change amount on each of the pixels by executing loop-C processing for each of the pixels within the processing target region.

In step S212, the blood vessel sharpness calculation unit 210 determines whether a processing target pixel is a pixel within the tubular region. In other words, the blood vessel sharpness calculation unit 210 determines whether the pixel is included in the blood vessel region. In a case where the pixel is within the tubular region (step S212: Yes), the reference region setting unit 112c sets (step S213) a range of pixels to be referred to in calculating the local absorbance change amount on the basis of the R-value on the processing target pixel (reference region). Specifically, the reference region is set such that the greater the R-value, that is, the shorter the imaging distance, the larger the reference region.

In subsequent step S214, the local absorbance change amount calculation unit 112 calculates a first eigenvalue (maximum eigenvalue) of the Hessian matrix by using the G/R-value calculated for the processing target pixel and the surrounding pixel within the reference region, and then, determines the first eigenvalue as a local absorbance change amount, namely, the blood vessel sharpness.

In contrast, in a case where it is determined in step S212 that the processing target pixel is not the pixel within the tubular region (step S212: No), the flowchart proceeds to the processing for the next pixel. With the loop C processing, blood vessel sharpness is calculated selectively for the pixel within the tubular region, among the pixels in the processing target region.

After the loop-C processing has been performed for all the pixels within the processing target region, operation of the computing unit 100 returns to the main routine.

As described above, according to the second embodiment, blood vessel sharpness is selectively calculated for the pixel within the tubular region, that is, the pixels within the blood vessel region, and blood vessel sharpness is not calculated for a non-blood vessel region. With this configuration, it is possible to further narrow abnormal candidate regions and thus to enhance accuracy in detecting abnormal regions.

### Third Embodiment

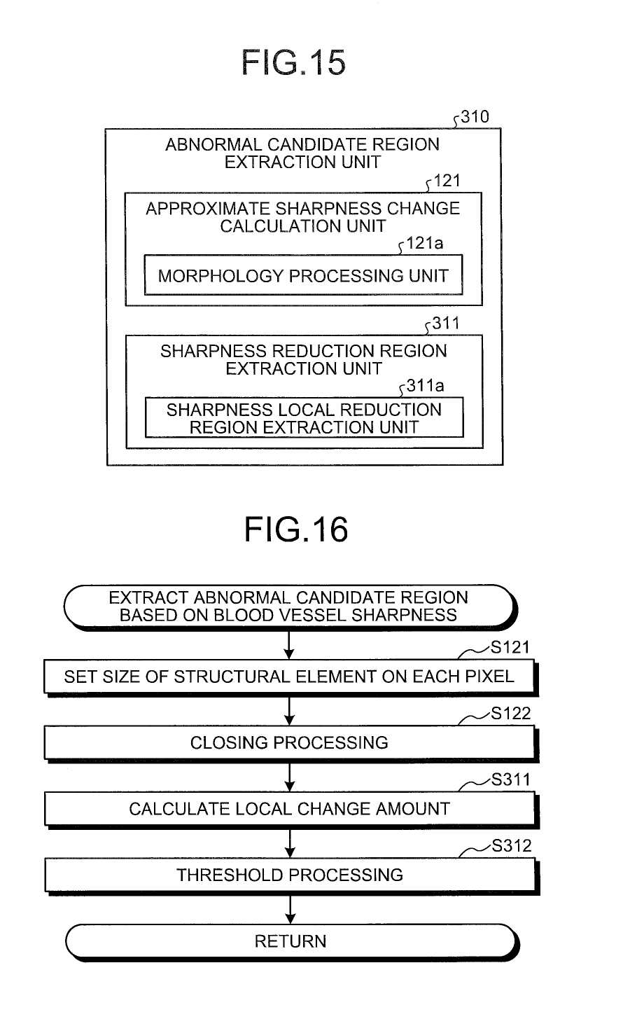

Next, a third embodiment of the present invention will be described. FIG. 15 is a block diagram illustrating a configuration of an abnormal candidate region extraction unit included in an image processing apparatus according to the third embodiment. On the image processing apparatus according to the third embodiment, the computing unit 100 includes an abnormal candidate region extraction unit 310 illustrated in FIG. 15 instead of the abnormal candidate region extraction unit 120. Note that individual configurations and operation of the computing unit 100 other than the abnormal candidate region extraction unit 310 and individual configurations and operation of the image processing apparatus 1 are similar to the case of the first embodiment.

The abnormal candidate region extraction unit 310 includes a sharpness reduction region extraction unit 311, instead of the sharpness reduction region extraction unit 122 illustrated in FIG. 1. The sharpness reduction region extraction unit 311 includes a sharpness local reduction region extraction unit 311a. The sharpness local reduction region extraction unit 311a calculates a local change for the approximate change in the blood vessel sharpness calculated by the approximate sharpness change calculation unit 121, and extracts a sharpness reduction region on the basis of the local change. With this, the sharpness reduction region extraction unit 311 extracts the region in which blood vessel sharpness has been locally reduced, as an abnormal candidate region.

Next, operation of the image processing apparatus according to a third embodiment will be described. General operation of the image processing apparatus according to the third embodiment is similar to the case of the first embodiment (refer to FIG. 2), except for a difference in details of processing in step S12 of extracting the abnormal candidate region, from the first embodiment.

FIG. 16 is a flowchart illustrating processing of extracting an abnormal candidate region, executed by the abnormal candidate region extraction unit 310. Note that steps S121 and S122 illustrated in FIG. 16 are similar to the steps in the first embodiment (refer to FIG. 6).

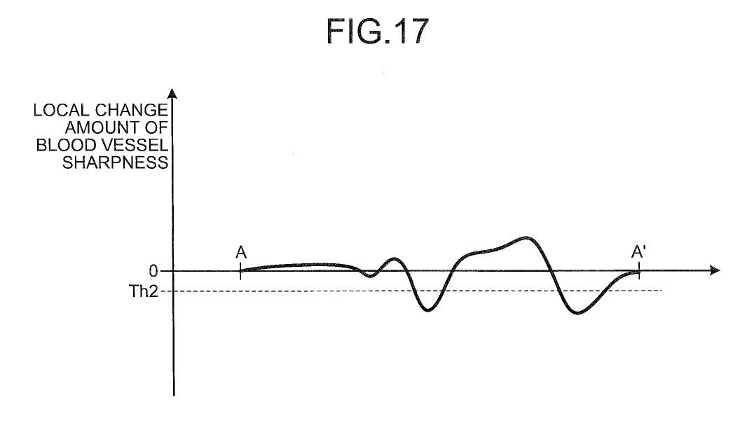

In step S311 subsequent to step S122, a sharpness local reduction region extraction unit 311a calculates a local change amount that is the local amount of change with respect to the approximate change in the blood vessel sharpness calculated in step S122. The method for calculating the local change amount is not particularly limited. Various known calculation methods can be applied. As one example, in the third embodiment, the local change amount is calculated using a band-pass filter. FIG. 17 is a graph illustrating a local change amount of blood vessel sharpness calculated for the approximate change in blood vessel sharpness, illustrated in FIG. 5.

In subsequent step S312, the sharpness reduction region extraction unit 311 performs threshold processing on the local change amount of blood vessel sharpness calculated in step S311 and extracts a region in which the local change amount is equal to or less than a predetermined threshold Th2, as an abnormal candidate region. As illustrated in FIG. 4, ordinary blood vessels exist around a region in which the visible vascular pattern is lost. Therefore, the region in which visible vascular pattern is lost is likely to appear as a region in which blood vessel sharpness has been locally reduced, as illustrated in FIG. 17. Accordingly, by performing threshold processing on the local change amount of blood vessel sharpness, it is possible to easily detect the region in which a visible vascular pattern is lost.

As described above, according to the third embodiment, since the local change amount is calculated for the approximate change in blood vessel sharpness, it is possible to selectively extract a region having a local change in sharpness, such as the region in which a visible vascular pattern is lost, as an abnormal candidate region. As a result, it is possible to enhance accuracy in detection of an abnormal region.

Note that in the third embodiment, it is also allowable to set the threshold (refer to step S312) to be used for threshold processing on the local change amount of blood vessel sharpness, for each of pixels on the basis of an R-value of the pixel, namely, imaging distance-related information, similarly to the modification example 1-1. Alternatively, similarly to the modification example 1-2, it would be allowable to set the threshold for each of the pixels on the basis of the coordinates of the pixel on the intraluminal image.

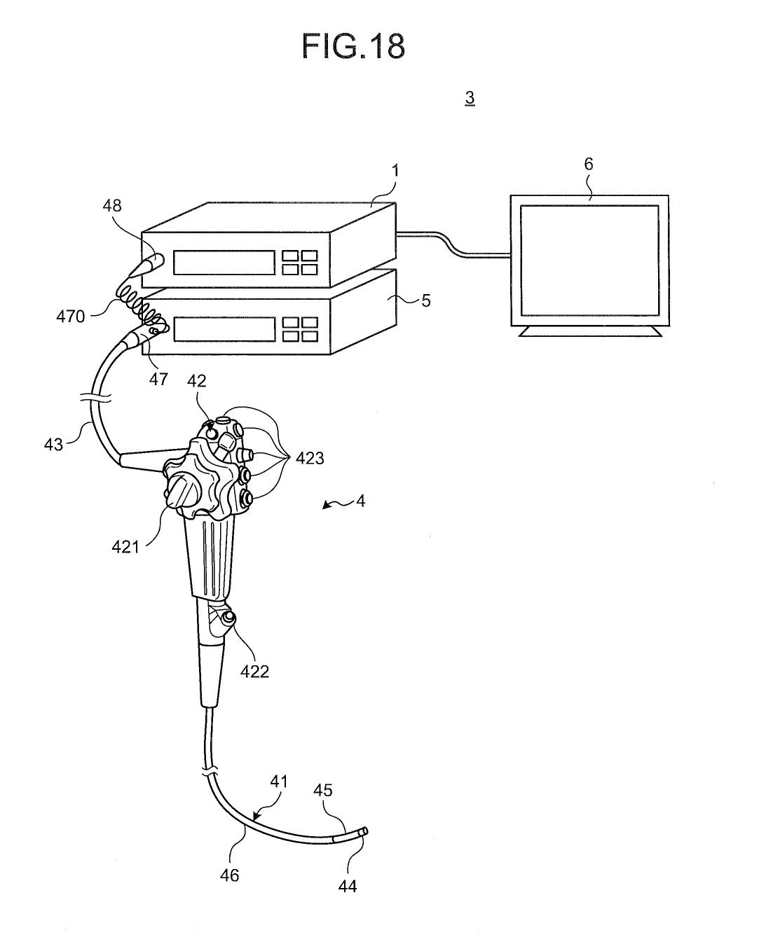

FIG. 18 is a diagram illustrating a general configuration of an endoscope system to which the image processing apparatus (refer to FIG. 1) according to the first embodiment of the present invention is applied. An endoscope system 3 illustrated in FIG. 18 includes the image processing apparatus 1, an endoscope 4, a light source device 5, and a display device 6. The endoscope 4 generates an image obtained by imaging the inside of the body of a subject by inserting its distal end portion into the lumen of the subject. The light source device 5 generates illumination light to be emitted from the distal end of the endoscope 4. The display device 6 displays an in-vivo image image-processed by the image processing apparatus 1. The image processing apparatus 1 performs predetermined image processing on the image generated by the endoscope 4, and together with this, integrally controls general operation of the endoscope system 3. Note that it is also allowable to employ the image processing apparatus described in the modification examples 1-1 to 1-3, or in the second and third embodiment, instead of the image processing apparatus 1.

The endoscope 4 includes an insertion unit 41, an operating unit 42, and a universal cord 43. The insertion unit 41 is a flexible and elongated portion. The operating unit 42 is connected on a proximal end of the insertion unit 41 and receives input of various operation signals. The universal cord 43 extends from the operating unit 42 in a direction opposite to the extending direction of the insertion unit 41, and incorporates various cables for connecting with the image processing apparatus 1 and the light source device 5.

The insertion unit 41 includes a distal end portion 44, a bending portion 45, and a flexible needle tube 46. The distal end portion 44 incorporates an image element. The bending portion 45 is a bendable portion formed with a plurality of bending pieces. The flexible needle tube 46 is long and flexible portion connected with a proximal end of the bending portion 45.

The image element receives external light, photoelectrically converts the light, and performs predetermined signal processing. The image element is implemented with a charge coupled device (CCD) image sensor and a complementary metal oxide semiconductor (CMOS) image sensor.

Between the operating unit 42 and the distal end portion 44, a cable assembly is connected. This cable assembly includes a plurality of signal lines arranged in a bundle, to be used for transmission and reception of electrical signals with the image processing apparatus 1. The plurality of signal lines includes a signal line for transmitting a video signal output from the image element to the image processing apparatus 1, and a signal line for transmitting a control signal output from the image processing apparatus 1 to the image element.

The operating unit 42 includes a bending knob 421, a treatment instrument insertion section 422, and a plurality of switches 423. The bending knob 421 is provided for bending the bending portion 45 in up-down directions, and in left-right directions. The treatment instrument insertion section 422 is provided for inserting treatment instruments such as a biological needle, biological forceps, a laser knife, and an examination probe. The plurality of switches 423 is an operation input unit for inputting operating instruction signals for not only the image processing apparatus 1 and the light source device 5, but also for peripheral equipment including an air feeding apparatus, a water feeding apparatus, and a gas feeding apparatus.

The universal cord 43 incorporates at least a light guide and a cable assembly. Moreover, the end portion of the side differing from the side linked to the operating unit 42 of the universal cord 43 includes a connector unit 47 and an electrical connector unit 48. The connector unit 47 is removably connected with the light source device 5. The electrical connector unit 48 is electrically connected with the connector unit 47 via a coil cable 470 having a coil shape, and is removably connected with the image processing apparatus 1.

The image processing apparatus 1 generates an intraluminal image to be displayed by the display device 6 on the basis of the image signal output from the distal end portion 44. The image processing apparatus 1 performs, for example, white balance processing, gain adjustment processing, γ correction processing, D/A conversion processing, and format change processing, and in addition to this, performs image processing of extracting an abnormal region from the above-described intraluminal image.

The light source device 5 includes a light source, a rotation filter, and a light source control unit, for example. The light source is configured with a white light-emitting diode (LED), a xenon lamp, or the like, and generates white light under the control of the light source control unit. The light generated from the light source is emitted from the tip of the distal end portion 44 via the light guide.

The display device 6 has a function of receiving an in-vivo image generated by the image processing apparatus 1 from the image processing apparatus 1 via the image cable and displaying the in-vivo image. The display device 6 is formed with, for example, liquid crystal, or organic electro luminescence (EL).

The above-described first to third embodiments and the modification examples of the embodiments can be implemented by executing an image processing program recorded in a recording device on a computer system such as a personal computer and a workstation. Furthermore, such a computer system may be used by connecting the computer system to another device including a computer system or a server via a local area network (LAN), a wide area network (WAN), or a public line such as the Internet. In this case, it is allowable to configure such that the image processing apparatus according to the first to third embodiments and the modification examples of the embodiments obtains image data of an intraluminal image via these networks, outputs a result of image processing to various output devices such as a viewer and a printer, connected through these networks, and stores the result of image processing in a storage device connected via these networks, such as a recording medium that is readable by a reading device connected via a network.

According to some embodiments, a candidate region for an abnormal region in which a visible vascular pattern is locally lost, is extracted based on sharpness of a visible vascular pattern in a mucosa region, and whether the candidate region is an abnormal region is determined based on a shape of the candidate region. With this feature, it is possible to detect, with high accuracy, a region in which the visible vascular pattern is locally lost in the intraluminal image.

The present invention is not limited to the first to third embodiments and the modification examples of the embodiments, but various inventions can be formed by appropriately combining a plurality of elements disclosed in the embodiments and the modification examples. For example, the invention may be formed by removing some elements from all the elements described in each of the embodiments and the modification examples, or may be formed by appropriately combining elements described in different embodiments and modification examples.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing apparatus comprising:
a blood vessel sharpness calculation unit configured to calculate blood vessel sharpness representing sharpness of a visible vascular pattern in a mucosa region in which a mucosa in a lumen is shown in an intraluminal image;
an abnormal candidate region extraction unit configured to extract a sharpness reduction region in which the blood vessel sharpness is reduced, as a candidate region for an abnormal region in which the visible vascular pattern is locally lost; and
an abnormal region determination unit configured to determine whether the candidate region is the abnormal region based on a shape of the candidate region.

2. The image processing apparatus according to claim 1,
wherein the blood vessel sharpness calculation unit comprises a local absorbance change amount calculation unit configured to calculate a local absorbance change amount of an absorbance wavelength component in the mucosa based on a pixel value of each pixel within the mucosa region, and
the blood vessel sharpness calculation unit is configured to output the local absorbance change amount as the blood vessel sharpness.

3. The image processing apparatus according to claim 2,
wherein the blood vessel sharpness calculation unit further comprises a region setting unit configured to set, as a calculation target region of the local absorbance change amount, a region obtained by eliminating at least one of a mucosa contour, a dark portion, specular reflection, a bubble, and a residue, from the intraluminal image.

4. The image processing apparatus according to claim 2,
wherein the local absorbance change amount calculation unit comprises:
an imaging distance-related information acquisition unit configured to acquire information related to an imaging distance between a subject shown in each pixel within the mucosa region and an imaging unit that has imaged the subject; and
a reference region setting unit configured to set a reference region to be referred to in calculating the local absorbance change amount, based on the information related to the imaging distance, wherein
the reference region setting unit is configured to narrow the reference region as the imaging distance is increased.

5. The image processing apparatus according to claim 2,
wherein the local absorbance change amount calculation unit comprises:
an imaging distance-related information acquisition unit configured to acquire information related to an imaging distance between a subject shown in each pixel within the mucosa region and an imaging unit that has imaged the subject; and
an absorbance wavelength component normalization unit configured to normalize a value of the absorbance wavelength component based on the information related to the imaging distance.

6. The image processing apparatus according to claim 1,
wherein the blood vessel sharpness calculation unit comprises a tubular region extraction unit configured to extract a tubular region based on a pixel value of each pixel within the mucosa region, and
the blood vessel sharpness calculation unit is configured to calculate a local absorbance change amount in the tubular region, as the blood vessel sharpness.

7. The image processing apparatus according to claim 1,
wherein the abnormal candidate region extraction unit comprises:
an approximate sharpness change calculation unit configured to calculate an approximate change in the blood vessel sharpness; and
a sharpness reduction region extraction unit configured to perform threshold processing on the approximate change, thereby to extract the sharpness reduction region.

8. The image processing apparatus according to claim 7,
wherein the approximate sharpness change calculation unit comprises a morphology processing unit configured to perform morphology processing on the blood vessel sharpness, and
the approximate sharpness change calculation unit is configured to calculate the approximate change in the blood vessel sharpness based on a result of the morphology processing.

9. The image processing apparatus according to claim 8,
wherein the morphology processing unit is configured to set at least a size of a structural element in morphology on each pixel based on information related to an imaging distance between a subject shown in each pixel within the mucosa region and an imaging unit that has imaged the subject.

10. The image processing apparatus according to claim 7,
wherein the sharpness reduction region extraction unit comprises:
an imaging distance-related information acquisition unit configured to acquire information related to an imaging distance between a subject shown in each pixel within the mucosa region and an imaging unit that has imaged the subject; and
a distance adaptive threshold setting unit configured to adaptively set a threshold to be used in the threshold processing, in accordance with the imaging distance corresponding to each pixel.

11. The image processing apparatus according to claim 10, further comprising a recording unit configured to record a plurality of types of information that associates the imaging distance with the threshold based on a depth of field defined in accordance with a focal length,
wherein the distance adaptive threshold setting unit is configured to set the threshold using one of the plurality of types of information corresponding to a focal length of an optical system included in the imaging unit that has imaged an inside of the lumen.

12. The image processing apparatus according to claim 7,
wherein the sharpness reduction region extraction unit comprises an optical system adaptive threshold setting unit configured to adaptively set a threshold to be used in the threshold processing, in accordance with characteristics of an optical system included in an imaging unit that has imaged an inside of the lumen.

13. The image processing apparatus according to claim 12,
wherein the optical system adaptive threshold setting unit is configured to set the threshold in accordance with a coordinate of each pixel within the mucosa region.

14. The image processing apparatus according to claim 7,
wherein the sharpness reduction region extraction unit comprises a sharpness local change amount calculation unit configured to calculate a local change amount in the approximate change in the blood vessel sharpness, and
the sharpness reduction region extraction unit is configured to extract the sharpness reduction region based on the local change amount.

15. The image processing apparatus according to claim 1,
wherein the abnormal region determination unit is configured to determine that the candidate region is the abnormal region if the candidate region has a certain degree of circularity.

16. The image processing apparatus according to claim 1,
wherein the abnormal region determination unit is configured to determine that the candidate region is the abnormal region if an area of the candidate region is equal to or less than a threshold.

17. An image processing method executed by an image processing apparatus for performing image processing on an intraluminal image, the method comprising:
calculating blood vessel sharpness representing sharpness of a visible vascular pattern in a mucosa region in which a mucosa in a lumen is shown in the intraluminal image;
extracting a sharpness reduction region in which the blood vessel sharpness is reduced, as a candidate region for an abnormal region in which the visible vascular pattern is locally lost; and
determining whether the candidate region is the abnormal region based on a shape of the candidate region.

18. A non-transitory computer-readable recording medium with an executable program stored thereon, the program causing a computer to execute:
calculating blood vessel sharpness representing sharpness of a visible vascular pattern in a mucosa region in which a mucosa in a lumen is shown in an intraluminal image;
extracting a sharpness reduction region in which the blood vessel sharpness is reduced, as a candidate region for an abnormal region in which the visible vascular pattern is locally lost; and
determining whether the candidate region is the abnormal region based on a shape of the candidate region.
